(19) United States
(12) Patent Application Publication
(10) Pub. No.: US 2005/0016964 A1
(43) Pub. Date: Jan. 27, 2005

(54) Device for destroying used syringe needles by melting

(76) Inventor: Yun-Ching Chuang, Taichung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/623,699

(22) Filed: Jul. 22, 2003

(51) Int. Cl. A61M 5/00

(52) U.S. Cl. 219/68

(57) ABSTRACT

A device for destroying used syringe needles includes a frequency converter disposed in a housing, and a coil disposed in the housing and connected to output terminals of the frequency converter at two ends so that the frequency converter can, when activated, power the coil with high frequency current for the coil to produce high temperatures; the coil having ring-shaped portions defining a middle hole under a through hole of an upper side of the housing for allowing needles to be passed into via the through hole; thus, used syringe needles can be melted and sterilized by heat of the coil when passed into the middle hole of the coil.

### BACKGROUND OF THE INVENTION

1. Field of the Invention

The present invention relates to a device for destroying used syringe needles, more particularly a device for melting used syringe needles with high temperatures.

2. Brief Description of the Prior Art

Needles of syringes have to be carefully disposed of after they are used, otherwise other people are likely to get pricked by the used needles, and infected with germs, diseases etc.

Referring to FIG. 6, a conventional machine 6 is used in factories for disposing of used syringe needles, which is comprised of motors, gears, and a transmission device; syringe needles 71 are passed between two opposing crushing gears 61 by means of the motors and transmission device so that the needles are broken into very small pieces, and plastic hollow barrels of the syringes are separated from the needles to be disposed of.

The above machine is found to have disadvantages as followings:
1. The machine is relatively large in size, and heavy in weight therefore it is only installed in factories for disposing of discarded objects. Consequently, after syringe needles are used in hospitals, staff of the hospitals has to collect the needles, and have the needles transported to factories specialized in destroying medical waste afterwards. And, the staff of hospitals and transportation is prone to get pricked by the needles in collecting or moving the needles, and get infected with germs and diseases. In addition, it will cause trouble to store the used needles before the needles are transported to factories.
2. The crushing gears 61 will get contaminated with germs due to contact with needles 71, and have to be regularly cleaned, and sterilized, costing much labor.

### SUMMARY OF THE INVENTION

It is a main object of the present invention to provide a device for destroying used syringe needles with high temperature so that needles can be easily and safely disposed of and right in the hospitals or clinics that use the needles.

The present device includes a frequency converter disposed in a housing, and a coil disposed in the housing and connected to output terminals of the frequency converter at two ends; when activated, the frequency converter can power the coil with high frequency current for the coil to produce high temperatures; the coil having ring-shaped portions defining a middle hole under a through hole of an upper side of the housing for allowing needles to be passed into via the through hole to be melted.

### BRIEF DESCRIPTION OF THE DRAWINGS

The present invention will be better understood by referring to the accompanying drawings, wherein:

FIG. 1 is a perspective view of the device for destroying used syringe needles according to the present invention,

FIG. 2 is a cross-sectional view of the device for destroying used syringe needles according to the present invention,

FIG. 3 is another cross-sectional view of the device for destroying used syringe needles according to the present invention,

FIG. 4 is a cross-sectional view of the device for destroying used syringe needles according to the second embodiment,

FIG. 5 is another cross-sectional view of the device for destroying used syringe needles according to the second embodiment, and

FIG. 6 is a partial plan of the conventional device for destroying used syringe needles as described in the Background.

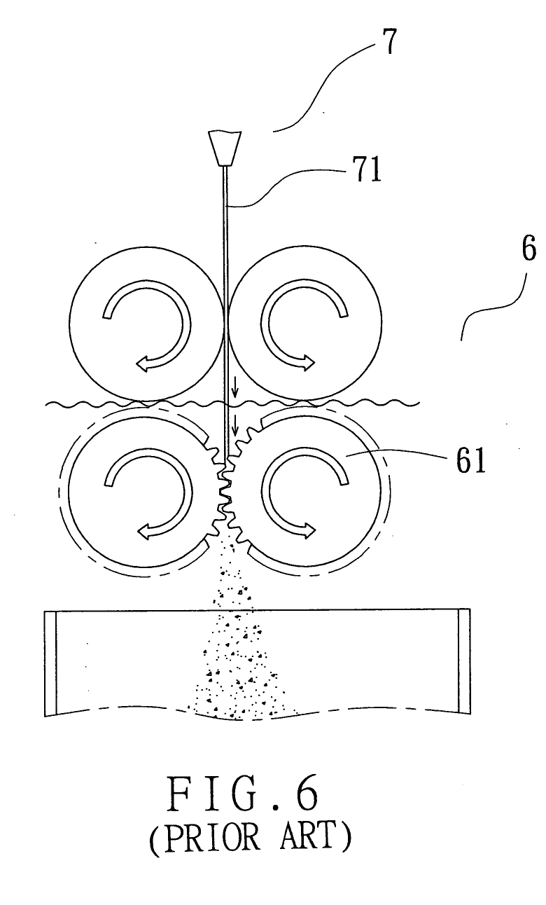

### DETAILED DESCRIPTION OF THE PREFERRED EMBODIMENTS

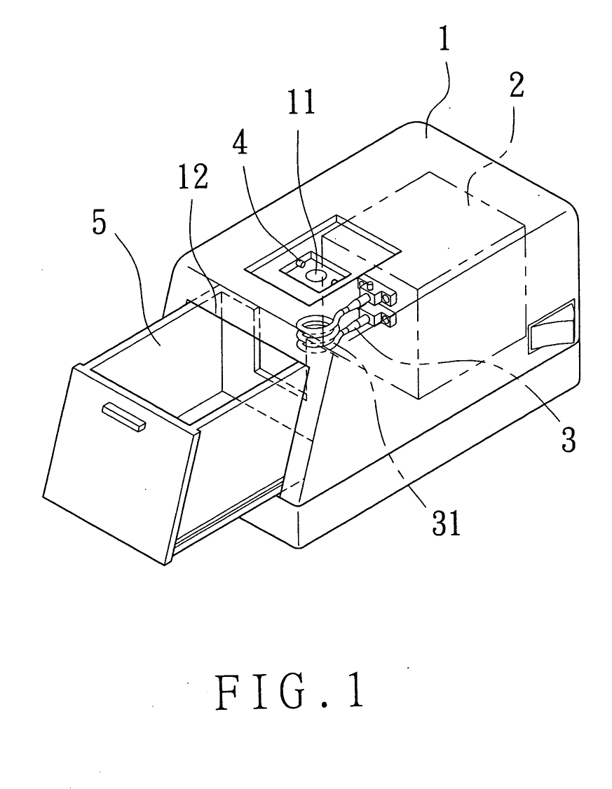

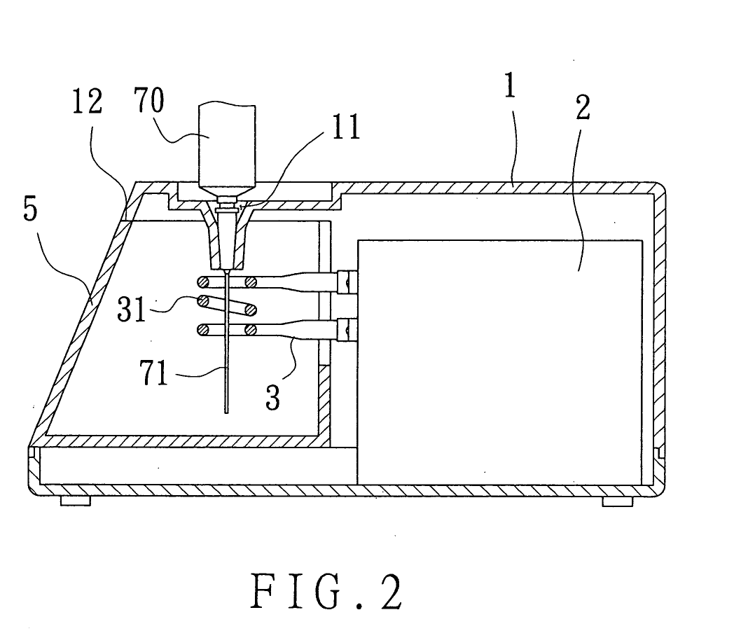

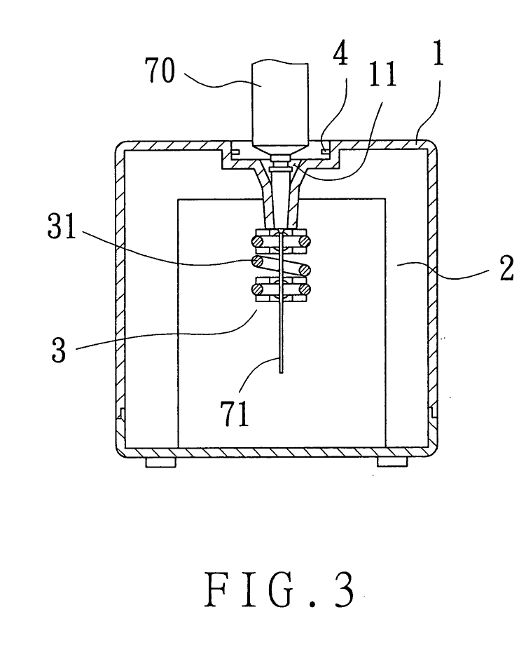

Referring to FIGS. 1, 2, and 3, a preferred embodiment of a device for destroying used syringe needles in the present invention includes a housing 1, a frequency converter 2, a coil 3, a sensor 4, and a collecting sink 5.

The housing 1 is formed with a through hole 11 on an upper side thereof for allowing used needles to be passed into, and an opening 12 on a lateral side. The collecting sink 5 can be in the form of a drawer, and is passed through the opening 12 to be right under the ring shaped portions 31 of the coil 3 for holding molten needles therein. The frequency converter 2 is disposed in the housing 1. The coil 3 is connected to output terminals of the frequency converter 2 at two ends thereof so that the frequency converter 2 can power the coil 3 with high frequency current for the coil 3 to produce high temperature when it is activated; a middle hole defined by ring shaped portions 31 thereof is right under, and aligned with, the through hole 11 of the housing 1 so that a needle can be passed into the middle hole of the coil 3 as well as the through hole 11. The sensor 4 is disposed in the housing 1, and is connected to the frequency converter 2 so that it can activate the frequency converter 2 when sensing presence of syringe needles.

To use the present device, the plastic hollow barrel 70 of a syringe is supported above the through hole 11, and the needle 71 of the syringe inserted into the middle hole of the coil 3 via the through hole 11 of the housing 1 so that the sensor 4 senses presence of the needle 71, and activates the frequency converter 2, and the coil 3 produce high temperature to melt the needle 71. Consequently, the needle 71 is melted, and falls onto the collecting sink 5.

The middle hole of the coil 3 can be shorter than the needle 71 in length, as shown in FIGS. 1 to 3; when the middle hole of the coil 3 is shorter than the needle 71, the sensor 4 can be set up such that it will activate the frequency converter 2 for the coil 3 to produce heat immediately after the needle 71 is passed through the upper end of the middle hole of the coil 3; thus, the whole needle 71 is melted, separated from the plastic hollow barrel 70 of the syringe, and drops onto the collecting sink 5.

When the middle hole of the coil 3 is shorter than the needle 71, and when the sensor 4 is set up such that it won't activate the frequency converter 2 for the coil 3 to produce heat until the needle 71 is passed through the lower end of the middle hole of the coil 3, the upper section of the needle 71 will be melted by the coil 3 but the lower section won't when the needle 71 is destroyed with the present device; finally, the lower section of the needle 71 will separate from the plastic hollow barrel 70 of the syringe, and drop onto the collecting sink 5 together with the molted upper section of the needle 71.

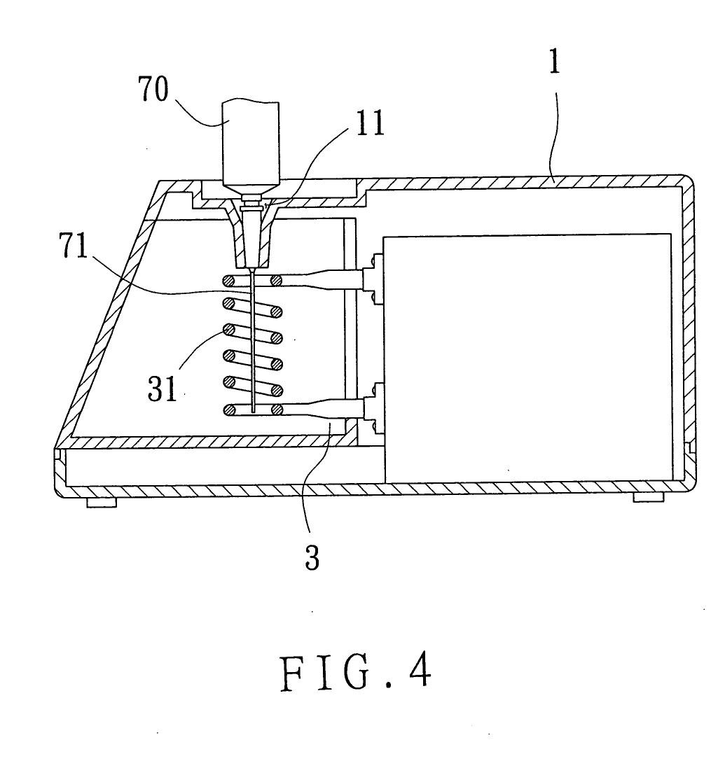

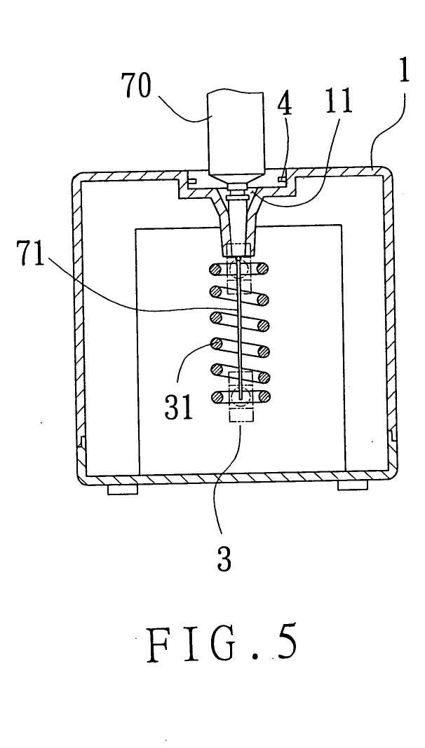

The middle hole of the coil 3 is longer than the needle 71 in length, as shown in FIGS. 4 to 5; when the middle hole of the coil 3 is longer than the needle 71, the whole needle 71 will be melted, separated from the plastic hollow barrel 70 of the syringe, and fall onto the collecting sink 5.

From the above description, it can be easily understood that the device for destroying used syringe needles in the present invention has advantages as followings:
1. The device is relatively compact therefore it is suitable for use in hospitals and clinics. Consequently, staff of hospitals or clinics doesn't have to collect used needles to have the needles transported to other locations specialized in destroying medical waste afterwards, eliminating the risk of people getting pricked by the needles.
2. Besides melting used needles, heat produced by the coil of the device can also kill bacteria on the needles.
3. With the sensor, the frequency converter 2 will make the coil 3 produce high temperature only when needles are passed into the through hole 11 of the housing 1.

1. A device for destroying used syringe needles, comprising
a frequency converter disposed in a housing; and
a coil connected to output terminals of the frequency converter at two ends so that the frequency converter can power the coil with high frequency current for the coil to produce high temperatures when activated; the coil having ring-shaped portions defining a middle hole under a through hole of an upper side of the housing for allowing needles to be passed into via the through hole;
whereby used syringe needles are melted and sterilized by heat produced by the coil after having been passed into the middle hole of the coil.

2. The device for destroying used syringe needles as claimed in claim 1, wherein a sensor is disposed in the housing, and connected to the frequency converter in order to activate the frequency converter when sensing presence of syringe needles in the middle hole of the coil.

3. The device for destroying used syringe needles as claimed in claim 1, wherein the housing has an opening on a lateral side thereof, and a collecting sink is passed into the housing via the opening and disposed under the ring-shaped portions of the coil for containing molten needles therein.
